(19) United States
(12) Patent Application Publication
(10) Pub. No.: US 2004/0170529 A1
(43) Pub. Date: Sep. 2, 2004

(54) Apparatus for the management of the physical state of a person in assisted self-treatment regimen

(76) Inventors: Pierangelo Bonini, Peschiera Borromeo, MI (US); Alberto Sanna, Milano (IT)

Correspondence Address: JACOBSON HOLMAN PLLC, 400 SEVENTH STREET N.W., SUITE 600, WASHINGTON, DC, 20004, US

(21) Appl. No.: 10/482,580

(22) PCT Filed: Jul. 1, 2002
(86) PCT No.: PCT/EP02/07290

(30) Foreign Application Priority Data
Jul. 1, 2001 (IT) MI2001A001517

(51) Int. Cl. G01N 33/00

(52) U.S. Cl. 422/68.1

(57) ABSTRACT

There is described an apparatus for the management of the physical state of a person in assisted self-treatment regimen which comprises: a) a computer (1) provided with a management software and at least a database for the recording of clinical rules and information on the physical state of the person; b) means (2) for the measurement of the physical state of the person connected with the computer (1); c) at least a reader (3) for the reading of codes of drugs, food integrators or other products, said reader (3) being connected with the computer (1); d) containers (4) with controlled access suitable to store drugs, integrators or other products; e) enablers (5) controlled by the computer (1) in order to enable the access to one or more containers (4) selected among said containers (4). The computer (1) is capable to control the enable (5) in response to the reading of the code of a drug or other on behalf of the reader (1), or in response to the combination of the information concerning the physical state of the person coming from the measurement means (2) and of the clinical rules.

The present invention concerns an apparatus for the management of the physical state of a person in assisted self-treatment regimen.

By person in assisted self-treatment regimen a patient under pharmacological treatment at home as also any person who, not being affected by various pathologies, wants to maintain his/her physical state in perfect efficiency is intended. For instance food diet, to quit smoking, gymnastics, etc.

Generally a person in assisted self-treatment regimen needs the periodic administration of several products, either pharmaceutical or not, at pre-established times. This is done in response to the physical state of the person, that must continuously be estimated in order to verify its improvement or worsening in such way that, according to its value, one can proceed with a higher or lower administration of products already in use or to the administration of different products.

Therefore, said person, besides being continuously careful to the observance of times and conditions for the administration of the prescribed products, must remember to purchase the products that are missing or about to finish and must necessarily undergo various examinations for the evaluation of his/her physical state. All this appears to be a somewhat onerous task for the person in assisted self-treatment regimen.

Scope of the present invention is to provide an apparatus for the management of the physical state of a person in assisted self-treatment regimen that allows to facilitate the carrying out of the aforesaid task.

According to the present invention such scope is attained by means of an apparatus for the management of the physical state of a person in assisted self-treatment regimen, characterised in that it comprises:

a) a computer provided with a management software and at least a database for the recording of clinical rules and information on the physical state of said person;

b) means for the measurement of the physical state of the person connected with said computer;

c) at least one reader for the reading of codes of drugs, food integrators or other products, said reader being connected with said computer;

d) containers with controlled access suitable to store said drugs, integrators or other products;

e) enablers controlled by said computer in order to enable the access to one or more containers selected among said containers;

said computer being able to control said enablers in response to the reading of the code of a drug or other on behalf of said reader, or in response to the combination of the information concerning the physical state of the person coming from said measurement means and of said clinical rules.

The characteristics and the advantages of the present invention will become evident from the following detailed description of an embodiment thereof, that is illustrated as a non-limiting example in the enclosed drawings, in which:

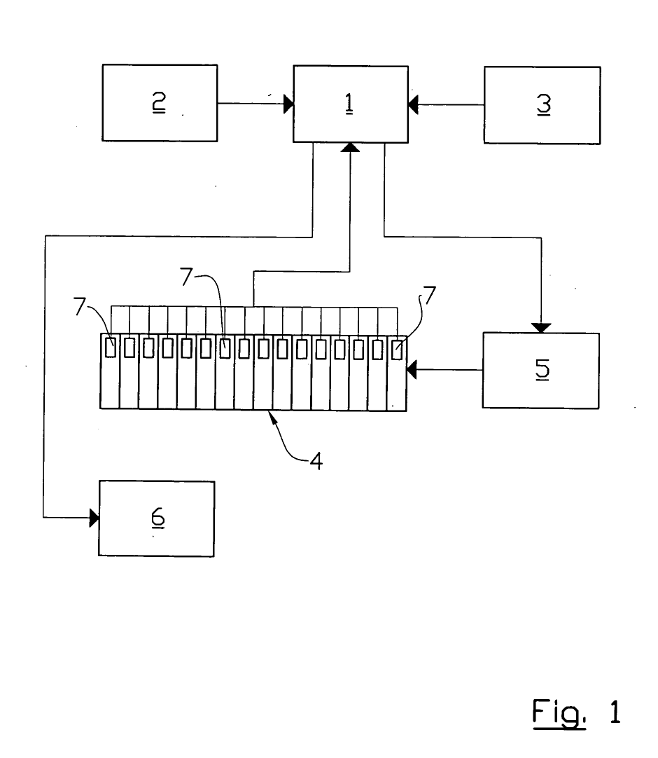

FIG. 1 is a schematic view of an apparatus for the management of the physical state of a person in assisted self-treatment regimen according to the present invention.

As schematically shown in FIG. 1, the apparatus according to the invention comprises a computer 1 provided with a management software and with a database in which there are contained information on the physical state of the person in assisted self-treatment regimen and clinical rules dictated by a physician or another health operator or even by a delegated relative or a therapist (as for instance trainer).

Such clinical rules can comprise medical, pharmacological, alimentary prescription and other.

A unit 2 for the measurement and evaluation of the physical state of the person is connected with the computer 1 through a wire or wireless transmission device; such unit 2 can comprise a meter of the pressure and of the vital parameters, an electrocardiograph, a blood test device, a scale or any other meter of the physical state. The measurement unit 2 sends to computer 1 the information regarding the physical state of the person in assisted self-treatment regimen together with the period of time in which the recording has been taken; such information is processed by the same computer 1 and input in the aforesaid database.

In addition a reader 3 for the reading of the drug or food integrators or other which must be stored in controlled access containers 4 is also connected with the computer 1, through a wire or wireless transmission device.

Enablers 5 are also connected with the computer 1 that are suitable to enable the access to one or more selected containers for the insertion or the withdrawal of a certain drug or integrator or other. The enabling can consist in the activation of a device for the opening of the container or, as an alternative, if the containers are provided with LED or sound devices, the enablers 5 can signal the exact container for the insertion or the withdrawal of the drug by means of light or sound signal.

Appropriate sensors 7 are provided for each container that are suitable to signal the access to the single containers 4. Said sensors are connected with the computer 1 for the recording of the access having taken place for the withdrawal or the insertion of the drug, integrator or other.

There is also provided a sound or optical device 6 connected with the computer 1 that emits alarm signals in the case in which the sensors 7 signal the access to a wrong container 4.

The operations that can be carried out by such apparatus are therefore those for the insertion and withdrawal of a drug or an integrator or other.

The operation of insertion of the drug or other takes place against the reading of the code of the drug or other by means of the reader 6 connected with the computer 1 to which the data relative to the aforesaid reading is sent. The computer 1 consequently controls the opening of one or more selected containers 4 through the enablers 5, or it signals the same selected containers 4 through light or sound signal for the opening on behalf of the user and the insertion of the drug or other. If a non-enabled container 4 is opened, the alarm device 6 signals it immediately.

The operation of withdrawal of the drug or other takes place against detection of the physical state of the person through the measurement unit 2 in a determined period of time; the information coming from the measurement unit 2 is sent to the computer 1. The latter according to the physical state and the detected clinical rules controls the enablers 5 for the opening of one or more selected containers 4 or it signals the same selected containers 4 through light or sound signal for the opening by the user and the withdrawal of the drug or other. The computer 1 memorises such opening by means of the appropriate sensors 7.

The computer 1 preferably comprises means to provide information on the outside, for instance to a physician or other health operator, and this preferably takes place by connection with other computers through the Internet or by telephone.

It is also possible that a drug or other whose code is read by the appropriate reader 2, is taken by the person in assisted self-treatment regimen without the insertion of the same in a container 4; in such case the administration of the drug or other will be recorded by the computer 1.

1. Apparatus for the management of the physical state of a person in assisted self-treatment regimen, characterised in that it comprises:
a) a computer (1) provided with a management software and at least a database for the recording of clinical rules and information on the physical state of said person;
b) means (2) for the measurement of the physical state of the person connected with said computer (1);
c) at least a reader (3) for the reading of codes of drugs, food integrators or other products, said reader (3) being connected with said computer (1);
d) containers (4) with controlled access suitable to store said drugs, integrators or other products;
e) enablers (5) controlled by said computer (1) in order to enable the access to one or more containers (4) selected among said containers (4);
said computer (1) being able to control said enablers (5) in response to the reading of the code of a drug or other by said reader part (1), or in response to the combination of the information concerning the physical state of the person coming from said measurement means (2) and of said clinical rules.

2. Apparatus according to claim 1, characterised in that said containers (4) are provided with sensors (7) connected with said computer (1), said sensors (7) being suitable to signal the opening of said containers (4) for the input or the withdrawal of said drug, integrator or other.

3. Apparatus according to claim 2, characterised in that it comprises an alarm device (6) connected with said computer (1) and suitable to emit alarm signals in the case in which said sensors (7) signal the opening of a non-enabled container (4).

4. Apparatus according to claim 1, characterised in that said computer (1) comprises means for the connection with external computers.

5. Apparatus according to claim 1, characterised in that said measurement means (2) send the information on the physical state of the person in assisted self-treatment regimen, together with the period of time in which the recording has been taken.
